(19) United States
(12) Patent Application Publication
(10) Pub. No.: US 2017/0095524 A1
(43) Pub. Date: Apr. 6, 2017

(54) METHODS FOR MODULATING CANCER CELLS AND STEM CELLS

(71) Applicant: The Scripps Research Institute, La Jolla, CA (US)

(72) Inventors: Peter W. VANDERKLISH, San Diego, CA (US); Christina SPEVAK, New York, NY (US)

(21) Appl. No.: 15/310,560

(22) PCT Filed: May 12, 2015
(86) PCT No.: PCT/US2015/030322

§ 371 Date: Nov. 11, 2016

Related U.S. Application Data
(60) Provisional application No. 61/991,745, filed on May 12, 2014.

(51) Int. Cl. A61K 38/05; A61K 31/277; A61K 31/519; A61K 31/53; A61K 31/365; A61K 31/506; A61K 31/5377; A61K 45/06; C07K 16/28

(52) U.S. Cl. CPC A61K 38/05; A61K 45/06; A61K 31/277; A61K 31/519; C07K 16/2863; A61K 31/365; A61K 31/506; A61K 31/5377; A61K 31/53; C07K 2317/76

(57) ABSTRACT

The present invention provides methods for modulating pluripotency of stem cells and proliferation of cancer cells. The modulation is achieved by promoting dephosphorylation of mRNA-binding protein 3 (RBM3) and down-regulating expression or cellular level of pluripotency factor LIN28. Related methods are provided in the invention for promoting differentiation of stem cells and for inhibiting growth of tumors in subjects. The therapeutic methods of the invention typically entail contacting with a target cell (e.g., a tumor cell) or administering to a subject harboring the target cell a calpain inhibitor and/or an IGF1R inhibitor. Some methods of the invention employ both a calpain inhibitor and an IGF 1R inhibitor.

### CROSS-REFERENCE TO RELATED APPLICATIONS

The subject patent application claims the benefit of priority to U.S. Provisional Patent Application No. 61/991,745 (filed May 12, 2014). The full disclosure of the priority application is incorporated herein by reference in its entirety and for all purposes.

### BACKGROUND OF THE INVENTION

Stem cells and other progenitor cells represent a promising approach in cell based therapeutics and regenerative medicine for treating various diseases and injuries. Stem cell transplantation holds the potential for repairing and regenerating damaged or injured tissues and organs. One of the challenges facing stem cell based therapeutics is how to regulate pluripotency of the stem cells and induce differentiation of stem cells in a desired and controlled manner.

Tumor recurrence after curative surgery remains a major obstacle for improving overall cancer survival, which may be in part due to the existence of cancer stem cells (CSC). Growing evidence suggests that human cancers are stem cell diseases and only a small subpopulation of cancer cells, endowed with stem cell-like features, might be responsible for tumor initiation, progression and chemoresistance. Cancer cells with the properties of stem cells possess the ability to self-renew, to undergo multilineage differentiation, and to survive an adverse tissue microenvironment. Current therapies target populations of rapidly growing and differentiated tumor cells, but have been shown to lack activity against CSCs.

There is a need in the art for better means for controlling pluripotency and differentiation of stem cells, as well as a need for more effective methods for treating cancers and preventing tumor recurrence. The present invention is directed to these and other needs.

### SUMMARY OF THE INVENTION

In one aspect, the invention provides methods for inhibiting phosphorylation, downregulating cellular level of phosphorylated form, or upregulating cellular level of dephosphorylated form, of mRNA-binding protein 3 (RBM3) in a target cell. The methods entail contacting the target cell with (1) a calpain inhibitor or (2) an IGF1R inhibitor, which leads to inhibition of phosphorylation, down-regulation of phosphorylated RBM3, or upregulation of de-phosphorylated RBM3 in the target cell. Some of the methods are specifically directed to cancer cells, cancer stem cells or stem cells. For example, the target cell can be hESC or iPSC.

In some embodiments, the target cell is present in a subject. In some embodiments, the target cell is contacted with both a calpain inhibitor and an IGF1R inhibitor. In some embodiments, the calpain inhibitor and the IGF1R inhibitor are small molecule compounds, antibodies or antigenic fragments. In some embodiments, the target cell is further contacted with a chemotherapeutic agent, an immunotherapeutic agent, or a metabolic agent.

In a related aspect, the invention provides methods for down-regulating expression or cellular level of pluripotency factor LIN28 in a target cell. These methods involve contacting the target cell with (1) a calpain inhibitor or (2) an IGF1R inhibitor, resulting in down-regulated expression or cellular level of pluripotency factor LIN28 in the target cell. In some of these methods, the target cell is a cancer cell, a cancer stem cell, or a stem cell (e.g., hESC or iPSC). In some methods, the target cell is present in a subject. In some methods, the target cell is contacted with both a calpain inhibitor and an IGF1R inhibitor. In some of these methods, the target cell is a cell which has shown reactivation of the Lin28 gene. In some of these methods, the calpain inhibitor and the IGF1R inhibitor are small molecule compounds, antibodies or antigenic fragments. In some of these methods, the target cell can be further contacted with a chemotherapeutic agent, an immunotherapeutic agent, or a metabolic agent.

In another aspect, the invention provides methods for treating or inhibiting the growth of a cancer in a subject. These methods entail administering to a subject in need of treatment a pharmaceutical composition comprising (1) a calpain inhibitor and/or (2) an IGF1R inhibitor, resulting in treatment or inhibition of the growth of a cancer in the subject. In some of these methods, the administered calpain inhibitor and IGF1R inhibitor are small molecule compounds, antibodies or antigenic fragments. In some methods, the subject is administered both a calpain inhibitor and an IGF1R inhibitor. In some of these methods, the calpain inhibitor and IGF1R inhibitor are administered in conjunction with one or more chemotherapeutic agents, immunotherapeutic agents, or metabolic agents. In some methods, the calpain inhibitor and the IGF1R inhibitor are administered prior to, simultaneously with, or subsequent to the chemotherapeutic agents, immunotherapeutic agents, or metabolic agents.

In another related aspect, the invention provides methods for inhibiting growth or proliferation, or inducing necrosis or apoptosis, of a target cell. Such methods involve contacting the cancer stem cell with a calpain inhibitor and an IGF1R inhibitor, which leads to inhibition of growth or proliferation, or induction of necrosis or apoptosis, of the target cell.

A further understanding of the nature and advantages of the present invention may be realized by reference to the remaining portions of the specification and claims.

### BRIEF DESCRIPTION OF THE DRAWINGS

FIGS. 1A-1B show miRNA biogenesis and a bi-stable switch controlling pluripotency. (A) Schematic of post-transcriptional steps in miRNA biogenesis and points where LIN28 and RBM3-2 of many RNA-BPs regulating biogenesis—act to modify miRNA maturation. LIN28 inhibits maturation of let-7 miRNAs, whereas RBM3 promotes it. (B) Illustration of reciprocal negative feedback between LIN28 and let-7 miRNAs. This pathway toggles between 2 bi-stable states, promoting either pluripotency and oncogenesis, or differentiation and cell cycle exit.

FIGS. 2A-2C show that RBM3 regulates the biogenesis of let-7 family miRNAs. (A) Northern blot showing pre-let-7i and mature let-7 in B104 cells under control (con) conditions and after siRNA-mediated knockdown (si) of RBM3. 5S RNA is a loading control. (B) Northern blots of similar experiments performed in HEK293 and HeLa cancer cell lines. (C) Western blot of the let-7 target k-ras in cells under control and RBM3 siRNA conditions. RBM3 suppression reduces let-7 and de-represses k-ras translation.

FIG. 3 shows that inhibition of calpain rescues expression of dephosphorylated forms of RBM3 in pluripotent cells. (Top) 2D Western blot of RBM3 in vehicle-treated (DMSO) P19 cells. Red arrows point to exogenously expressed mutant of RBM3 (A4F) with 4 C-term Y to F mutations, and to two phosphorylated endogenous isoforms of RBM3 that differ by a single arginine (Arg). (Bottom) Western showing a shift in RBM3 pI distribution after incubation of P19 cells with a calpain inhibitor (Calpeptin). A basic spot not present in the control condition appears that corresponds to the pI of dephosphorylated Arg+RBM3, the form of RBM3 that preferentially associates with pre-miRNAs.

FIGS. 4A-4B show regulation of LIN28 levels by RBM3, IGF1R, and calpain. (A) Western blot showing the effect of RBM3 knock-down (siRNA) on LIN28 levels in P19 cells treated at the neurosphere stage with (or without) retinoic acid (RA) to induce neural differentiation. RBM3 knock-down increases LIN28 and blocks the ability of RA to decrease LIN28. (B) Western blots showing reduction of LIN28 in P19 cells following acute treatment (˜12 hrs) with inhibitors of IGF1R and calpain (each of which increases the expression of the dephosphorylated form of RBM3). (left panels) Treatment with the IGF1R inhibitor picropodophyllin (PPP, 10 nM) reduces LIN28 levels. (right panels) Similarly, treatment with the calpain inhibitor calpeptin (Cpep, 50 μM) reduces LIN28 levels and this is enhanced by co-treatment with PPP. HDAC is a loading control.

FIGS. 5A-4D show that RBM3 is a substrate of Calpain in vitro. (A & B) Purified recombinant myc-RBM3 was incubated with purified calpain 1 and Ca⁺⁺ and samples were taken at the indicated times for Western blot analysis of RBM3 levels using antibodies recognizing the N-terminal myc epitope tag (A) or for the c-terminus (B). A C-terminal breakdown product (BDP) is evident in B. (C & D) Similar experiment using cytoplasmic lysates of B104 cells expressing myc-RBM3. N-terminal BDPs are seen in C, whereas C-terminal BDPs are evident in D.

FIGS. 6A and 6B show that the phosphorylation state of RBM3 modulates its sensitivity to cleavage by calpain in vitro. (A) B104 cells were treated with IGF1 prior to lysis and addition of purified calpain 1. Western blots show myc-RBM3 and endogenous RBM3 at indicated times with calpain. (B) Purified myc-RBM3 was treated with phosphatase in vitro before incubation with calpain 1. Western blots show myc-RBM3 in cleavage reactions at the indicated time points.

FIGS. 7A-7D show results from additional studies of reducing LIN28 levels via synergistic dual inhibitor strategies. (A & B) Graphs of changes in RBM3 and LIN28 expression in P19 cells treated with the IGF1R inhibitor PPP (10 nM), the calpain inhibitor calpeptin (Cpep, 50 μM), and a combination of PPP plus Cpep (A: n=10-14; B: n=20-24). (C) (top panels) Example of Westerns of LIN28 in P19 cells exposed to PPP, Cpep, and PPP+Cpep; (bottom panels) Example of Westerns of LIN28 and RBM3 in samples from P19 cells treated in triplicate with control and Cpep+PPP conditions. (D) Fold change in let-7 family miRNAs and miR-21 in P19 cells treated with PPP+Cpep as measured by qRT-PCR (n=4). All treatments were for 36 hours. It was found from the same studies that proteasome inhibitors did not change LIN28 levels over same period; neither did caspase or cathepsin inhibitors (data not shown). * p<0.05 vs control, 2-tailed t-test; ** p<0.05 Cpep+PPP vs PPP and Cpep+PPP vs Cpep.

FIGS. 8A and 8B show a graphical illustration of a novel method to reduce LIN28: (A) High LIN28 levels contribute to pluripotency and oncogenesis by suppressing the biogenesis of let-7 miRNAs. A dephosphorylated form of RBM3 greatly enhances the biogenesis of let-7 and other miRNAs that promote differentiation and downregulate LIN28, but this effect is suppressed in pluripotent and cancerous cells by IGF1R-mediated tyrosine phosphorylation (upon binding of IGF1, IGFII, or insulin) and Calpain-mediated cleavage of dephosphorylated RBM3. (B) Strategy of using inhibitors of IGF1R and Calpain (or antibodies to IGF1R) to promote accumulation of the dephosphorylated form of RBM3 in pluripotent and cancerous cells. This suppresses LIN28 via upregulation of let-7 and other miRNAs, which then leads to cell cycle exit, differentiation, and, in the case of cancer stem cells, heightened sensitivity to chemotherapeutics.

### DETAILED DESCRIPTION OF THE INVENTION

### I. Introduction

The present invention is predicated in part on the discovery by the present inventors of a novel, pharmacological approach to modulate RNA-binding motif protein 3 (RBM3) and pluripotency factor LIN28 levels in cancers and stem cells. High LIN28 levels contribute to pluripotency and oncogenesis by suppressing the biogenesis of let-7 miRNAs. RNA-binding motif protein 3 (RBM3) promotes the biogenesis of let-7 family and other microRNAs (miRNAs) that negatively regulate LIN28 synthesis. As detailed herein, a dephosphorylated form of RBM3 greatly enhances the biogenesis of let-7 and other miRNAs that promote differentiation and downregulate LIN28. The inventors found that RBM3 is regulated by two signaling pathways involved in the maintenance of pluripotency and oncogenesis: the Insulin-Like Growth Factor 1 Receptor (IGF1R) and the Ca⁺⁺-activated cysteine protease, Calpain. The activity of each of these enzymes reduces the abundance of a de-phosphorylated form of RBM3 that mediates its effects on miRNA biogenesis. It was further revealed by the inventors that RBM3 is a substrate of Calpain, and that the phosphorylation state of RBM3 modulates its sensitivity to cleavage by calpain in vitro.

The present invention is directed to the use of inhibitors of IGF1R and calpain to promote accumulation of the dephosphorylated form of RBM3 in pluripotent and cancerous cells. This suppresses LIN28 via upregulation of let-7 and other miRNAs, which then leads to cell cycle exit, differentiation, and, in the case of cancer stem cells, heightened sensitivity to chemotherapeutics. Some embodiments of the invention employ a strategy of dual inhibition of IGF1R and Calpain, in a synergistic manner (as exemplified herein), to modulate RBM3 phosphorylation and LIN28 cellular level. For example, the inhibitors can be used to inhibit RBM3 phosphorylation (or to up-regulate de-phosphorylated RBM3) and/or to reduce LIN28 expression or cellular level in cancers and “cancer stem cells”, as well as in human Embryonic Stem Cells (hESCs), induced Pluripotent Stem Cells (iPSCs), and endogenous stem cells during disease and after injury. In some other embodiments, individual inhibition of one of the two pathways is employed to effectively down-regulate LIN28 expression in cancer stem cells, which has not been previously demonstrated successfully in the art.

The methods of the invention could find broad use in cancer therapy as an add-on to chemotherapeutics, or as neoadjuvant and adjuvant therapy. 15% of all cancers (including the most frequent—breast, prostate and lung cancers) exhibit reactivation of LIN28. Importantly, cancer stem cells thought to seed tumor formation and mediate recurrence in potentially all cancer types express high levels of LIN28, which contributes directly to their stem cell phenotype and resistance to chemotherapy. In addition, pharmacological reduction of LIN28 can provide a useful tool in regenerative medicine applications of hESCs and iPSCs, e.g., aid in the temporal and lineage control of stem cells.

The following sections provide more detailed guidance for practicing the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

A calpain is a protein belonging to the family of calcium-dependent, non-lysosomal cysteine proteases (proteolytic enzymes) expressed ubiquitously in mammals and many other organisms. Calpains constitute the C2 family of protease clan CA in the MEROPS database. The calpain proteolytic system includes the calpain proteases, the small regulatory subunit CAPNS1, also known as CAPN4, and the endogenous calpain-specific inhibitor, calpastatin.

The calpains are a conserved family of cysteine proteinases that catalyze the controlled proteolysis of many specific substrates. Calpain activity is implicated in several fundamental physiological processes, including cytoskeletal remodeling, cellular signaling, apoptosis and cell survival. Calpain expression is altered during tumorigenesis, and the proteolysis of numerous substrates, such as inhibitors of nuclear factor-κB (IκB), focal adhesion proteins and proto-oncogenes (for example, MYC), has been implicated in tumor pathogenesis. There are currently 14 known human calpain isoform genes, which are defined by the presence of a protease domain that is similar to that found in micro (μ)-calpain, which is one of the two most extensively studied isoforms, the other being milli (m)-calpain. Although many of the precise physiological functions of the calpain isoforms and mechanisms controlling proteolytic activity remain to be fully elucidated, experimental studies have demonstrated clear roles for calpains in a number of important cellular processes, including cell motility and apoptosis.

The archetypical members of the calpain family, μ-calpain and m-calpain, which were named on the basis of the concentration of calcium ions required for their activity in vitro, require calcium and a neutral pH for proteolytic activity. Both μ-calpain and m-calpain are heterodimers consisting of a catalytic (80 kDa) subunit and a regulatory subunit (28 kDa). The catalytic subunits differ between μ-calpain and m-calpain and are formed by calpain 1 (encoded by CAPN1) and calpain 2 (encoded by CAPN2), respectively. The regulatory subunit is common to both isoforms and is encoded by CAPNS1. The catalytic and regulatory subunits have four (DI to DIV) and two (DV and DVI) domains, respectively. DI is autolysed when calpains are activated by calcium, but this does not seem to be a prerequisite for activation. DII, the conserved protease domain, is divided into the subdomains IIa and IIb, which, on binding calcium, form a signal domain (II) that contains the catalytic cleft. DIII contains characteristic C2 domains and is involved in structural changes during calcium binding. The carboxy-terminal domains DIV (catalytic subunit) and DVI (regulatory subunit) contain five EF hands, not all of which are involved in binding calcium, as the fifth EF hand aids dimerization of the subunits. DVI at the amino-terminal of the regulatory subunit contains a string of glycine residues that may enable interaction with the plasma membrane and are autolysed during calpain activation.

Cancer stem cells (CSCs) refer to a subset of tumor cells that has the ability to self-renew. They are termed cancer stem cells to reflect their stem cell-like properties. CSCs are cancer cells (found within tumors or hematological cancers) that possess characteristics associated with normal stem cells, specifically the ability to give rise to all cell types found in a particular cancer sample. CSCs are therefore tumorigenic (tumor-forming), perhaps in contrast to other non-tumorigenic cancer cells. CSCs may generate tumors through the stem cell processes of self-renewal and differentiation into multiple cell types. Such cells are proposed to persist in tumors as a distinct population and cause relapse and metastasis by giving rise to new tumors.

Administration “in conjunction with” one or more other therapeutic agents includes simultaneous (concurrent) and consecutive administration in any order.

The term “contacting” has its normal meaning and refers to combining two or more agents (e.g., polypeptides or small molecule compounds) or combining agents and cells. Contacting can occur in vitro, e.g., combining two or more agents or combining an agent and a cell in a test tube or other container. Contacting can also occur in a cell or in situ, e.g., contacting two polypeptides in a cell by coexpression in the cell of recombinant polynucleotides encoding the two polypeptides, or in a cell lysate. Contacting can also occur inside the body of a subject, e.g., by administering to the subject an agent which then interacts with the intended target (e.g., a tissue or a cell).

The Insulin-like Growth Factor 1 (IGF-1) receptor is a protein found on the surface of human cells. It is a transmembrane receptor that is activated by a hormone called Insulin-like growth factor 1 (IGF-1) and by a related hormone called IGF-2. It belongs to the large class of tyrosine kinase receptors. This receptor mediates the effects of IGF-1, which is a polypeptide protein hormone similar in molecular structure to insulin. IGF-1 plays an important role in growth and continues to have anabolic effects in adults—meaning that it can induce hypertrophy of skeletal muscle and other target tissues. Mice lacking the IGF-1 receptor die late in development, and show a dramatic reduction in body mass, testifying to the strong growth-promoting effect of this receptor. Mice carrying only one functional copy of IGF1R are normal, but exhibit a ˜15% decrease in body mass.

Two alpha subunits and two beta subunits make up the IGF-1 receptor. Both the α and subunits are synthesized from a single mRNA precursor. The precursor is then glycosylated, proteolytically cleaved, and crosslinked by cysteine bonds to form a functional transmembrane αβ chain. The α chains are located extracellularly while the β subunit spans the membrane and are responsible for intracellular signal transduction upon ligand stimulation. The mature IGF-IR has a molecular weight of approximately 320 kDa. The receptor is a member of a family which consists of the Insulin Receptor and the IGF-2R (and their respective ligands IGF-1 and IGF-2), along with several IGF-binding proteins.

IGF1R and IR both have a binding site for ATP, which is used to provide the phosphates for autophosphorylation. There is a 60% homology between IGF1R and the insulin receptor. In response to ligand binding, the α chains induce the tyrosine autophosphorylation of the chains. This event triggers a cascade of intracellular signaling that, while somewhat cell type specific, often promotes cell survival and cell proliferation.

When used herein the terms “miR” and “miRNA” are used to refer to microRNA, a class of small RNA molecules that are capable of modulating RNA translation (see, Zeng and Cullen, RNA, 9:112-123, 2003; Kidner and Martienssen Trends Genet, 19:13-6, 2003; Dennis C, Nature, 420:732, 2002; and Couzin J, Science 298:2296-7, 2002). MicroRNAs (miRNAs) encompass a family of ˜22 nucleotide (nt) non-coding RNAs. These RNAs have been identified in organisms ranging from nematodes to humans. Many miRNAs are evolutionarily conserved widely across phyla, regulating gene expression by post-transcriptional gene repression. The long primary transcripts (pri-miRNAs) are transcribed by RNA polymerase II; processed by a nuclear enzyme Drosha; and released as a ˜60 bp hairpin precursor (pre-miRNAs). Pre-miRNAs are processed by RNase III enzymes, Dicer, to ˜22 nt (mature miRNAs) and then incorporated into RISC (RNA-induced silencing complex). The complex of miRNAs-RISC binds the 3′ UTR of the target mRNAs and conducts translational repression or degradation of mRNAs.

LIN28 is a pluripotency factor implicated in cell pluripotency, reprogramming, and oncogenesis. Encoded by the Lin-28 gene (Moss et al., Dev. Biol. 258: 432-42, 2003), human LIN28 is a conserved RNA-binding protein microRNA-binding protein that binds to and enhances the translation of the IGF-2 (insulin-like growth factor 2) mRNA. LIN28 has also been shown to bind to the let-7 pre-microRNA and block production of the mature let-7 microRNA in mouse embryonic stem cells. In pluripotent embryonal carcinoma cells, LIN28 is localized in the ribosomes, P-bodies and stress granules.

LIN28 is thought to regulate the self-renewal of stem cells. In nematodes, the LIN28 homolog lin-28 is a heterochronic gene that determines the onset of early larval stages of developmental events in Caenorhabditis elegans, by regulating the self-renewal of nematode stem cells in the skin (called seam cells) and vulva (called VPCs) during development. In mice, LIN28 is highly expressed in mouse embryonic stem cells and during early embryogenesis. LIN28 is also highly expressed in human embryonic stem cells and has been used to enhance the efficiency of the formation of induced pluripotent stem (iPS) cells from human fibroblasts.

Regenerative treatment or medicine is the “process of replacing or regenerating human cells, tissues or organs to restore or establish normal function”. This field holds the promise of regenerating damaged tissues and organs in the body by replacing damaged tissue and/or by stimulating the body's own repair mechanisms to heal previously irreparable tissues or organs. Regenerative medicine also empowers scientists to grow tissues and organs in the laboratory and safely implant them when the body cannot heal itself. Regenerative medicine treatment refers to a group of biomedical approaches to clinical therapies that may involve the use of stem cells. See, e.g., Riazi et al., Methods Mol. Biol. 482: 55-90, 2009. Examples include the injection of stem cells or progenitor cells (cell therapies); the induction of regeneration by biologically active molecules administered alone or as a secretion by infused cells (immunomodulation therapy); and transplantation of in vitro grown organs and tissues (tissue engineering).

The term “subject” for purposes of treatment refers to any animal classified as a mammal, e.g., human and non-human mammals. Examples of non-human animals include dogs, cats, cattle, horses, sheep, pigs, goats, rabbits, and etc. Unless otherwise noted, the terms “patient” or “subject” are used herein interchangeably. Preferably, the subject is human.

The term “treating” or “alleviating” includes the administration of compounds or agents to a subject to prevent or delay the onset of the symptoms, complications, or biochemical indicia of a disease (e.g., a cancer), reducing the symptoms or arresting or inhibiting further development of the disease, condition, or disorder. Subjects in need of treatment include those already suffering from the disease or disorder as well as those being at risk of developing the disorder. Treatment may be prophylactic (to prevent or delay the onset of the disease, or to prevent the manifestation of clinical or subclinical symptoms thereof) or therapeutic suppression or alleviation of symptoms after the manifestation of the disease. In the treatment of an inflammatory disease or disorder, a therapeutic agent may directly decrease the pathology of the disease, or render the disease more susceptible to treatment by other therapeutic agents.

### III. Inhibiting IGF1R and Calpain to Promote RBM3 Dephosphorylation and LIN28 Downregulation

The invention provides methods for modulating (e.g., in a titratable way) mRNA-binding protein 3 (RBM3) in target cells (e.g., tumor cells or stem cells). The modulation typically relates to inhibiting phosphorylation (or promoting dephosphorylation), or to downregulating cellular level of phosphorylated form (or upregulating cellular level of dephosphorylated form), of RBM3 in the target cells. De-phosphorylated form of RBM3 enhances the biogenesis of let-7 family and other microRNAs (miRNAs) that negatively regulate synthesis of pluripotency factor LIN28. In related embodiments, the invention provides methods for down-regulating expression or cellular level of LIN28 in target cells (e.g., cancer cells that have shown reactivation of the Lin28 gene). As detailed herein, LIN28 is pro-oncogenic and implicated in cell pluripotency. High levels of LIN28 contribute directly to cancer stem cell phenotype and resistance to chemotherapy. In some other related embodiments, the invention provides methods for inhibiting the growth or proliferation of a target cell (e.g., a tumor cell or a cancer stem cell). In some other related embodiments, the invention provides method for treating or inhibiting the growth of a cancer in a subject. Additional methods are provided in the invention for treating or inhibiting the growth of a cancer, or for promoting differentiation of stem cells (e.g., hESC, iPSC or endogenous stem cells) in a subject. Some related embodiments are directed to methods of inducing necrosis or apoptosis of cancer cell and/or cancer stem cells in a subject, using inhibitors of calpain and IGF1R.

The methods of the invention typically involve contacting the target cell (e.g., a tumor or cancer stem cell) with, or administering to the subject afflicted with a tumor or otherwise in need of treatment, a composition that contains a calpain inhibitor and/or an IGF1R inhibitor of IGF1R. In some methods, the dual inhibitors are contacted with a target cell or co-administered to a subject in conjunction with another therapeutic agent (e.g., a chemotherapeutic agent, an immunotherapeutic agent, or a metabolic agent as detailed below). In the practice of the invention, an additional step of measuring expression or cellular level of LIN28 or dephosphorylated RBM3 in the target cell can be included. The expression or cellular level of these molecules in the treated cell can then be compared to that of a control cell. The control cell can be the same type of cell but which is not treated with the inhibitors. Expression or cellular levels of these molecules in the target cells or control cells can be readily determined with methods exemplified herein (e.g., western blot) or other standard procedures well known in the art or exemplified herein.

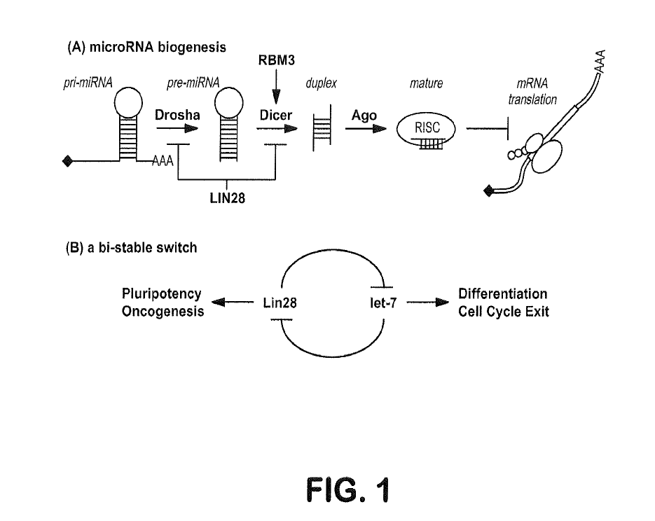

miRNAs are a family of ˜22nt non-coding RNAs that regulate mRNA translation. There are ˜2000 miRNAs in humans, many of which play critical roles in development and differentiation, and in a wide spectrum of diseases, including cancer. Most miRNAs are produced via a two-step cleavage process whereby ˜70nt stem-loop precursors (pre-miRNAs) are excised from primary transcripts (pri-miRNAs) by Drosha in the nucleus, followed by Dicer-mediated excision of ˜22nt duplexes in the cytoplasm. Typically, one strand of a duplex is integrated into the RNA-induced Silencing Complex (RISC) where translational regulation is guided by sequence complementarity between miRNAs and mRNAs. Importantly, these steps in miRNA biogenesis are key points of regulation where RNA-binding proteins (RNA-BPs) can influence the rate and profile of miRNA production (FIG. 1A). The let-7 family of miRNAs (12 in mammals) plays a major role in cell fate determination by negatively regulating the translation of various “stemness” or “dedifferentiation” factors and cell cycle regulators.

The role of LIN28 in maintaining pluripotency through the regulation of miRNA biogenesis and the role of this pathway in various cancers and stem cell differentiation have been well documented in the literature. The biogenesis of let-7 family members is blocked at both the Drosha and Dicer steps by the pluripotency factor LIN28, an RNA-BP that is itself negatively regulated by let-7 at the mRNA level. LIN28 is highly expressed in ESCs where it maintains pluripotency, and is sufficient to produce “induced Pluripotent Stem Cells” (iPSCs) when ectopically expressed with other factors. The reciprocal negative influences between LIN28 and let-7 family members have been proposed to constitute a “bi-stable switch” regulating pluripotency and differentiation, e.g., determining whether stem cells (and “cancer stem cells”) remain pluripotent or differentiate (FIG. 1B).

LIN28 is upregulated in about 15% of cancers and promotes cell transformation by enabling the expression of other oncogenes that are normally repressed by let-7. In several cancers, the expression level of LIN28 correlates strongly with recurrence rates. Interestingly, high LIN28 expression defines a subpopulation of cells termed “cancer stem cells” that seed tumor formation in potentially all cancers. These cells are particularly resistant to chemotherapy and this has been linked to LIN28. The pro-oncogenic and disease exacerbating effects of LIN28 are clearly documented in breast cancers, the second most frequent cancer in the US. Germ line mutations in the let-7 binding site in LIN28 mRNA increase susceptibility to breast cancer. In addition, LIN28 promotes expression of the Her2 oncogene that is associated with most breast cancers, and mediates resistance to Paclitaxel and radiotherapy. High LIN28 levels appear to also promote tumor genesis in Her2-breast cancers. Finally, LIN28 induction is part of a positive feedback circuit linking inflammatory signaling (IL-6), let-7 suppression, and oncogenesis in breast cancer.

Enhancing RBM3 dephosphorylation and thereby lifting the LIN28-mediated suppression of let-7 biogenesis would promote differentiation and chemotherapeutic sensitivity in cancer cells and cancer stem cells. In addition, manipulating LIN28 expression would be of broad utility in regenerative medicine where it could be part of a strategy to guide the differentiation of introduced hESC or iPSC (or even endogenous stem cells) along particular paths or at particular times during disease or following injury. Currently, no effective and therapeutically applicable approach for reducing LIN28 levels has actually been demonstrated. Thus, there are unmet needs in the art for better and more effective means for reducing LIN28 expression.

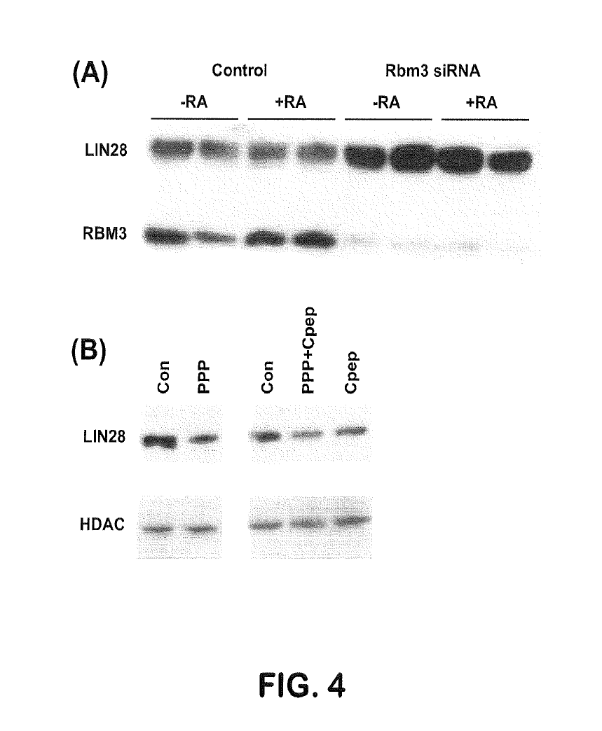

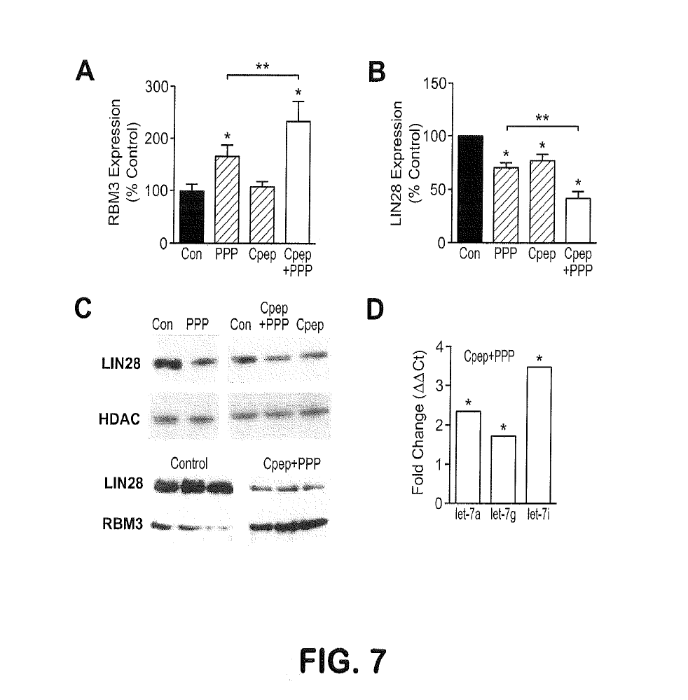

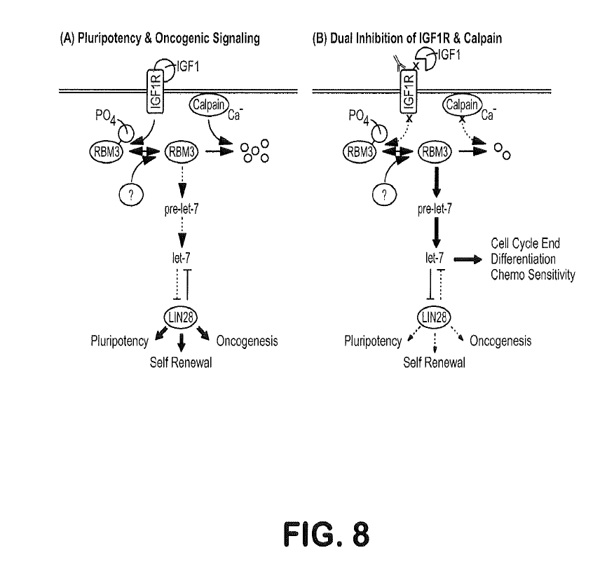

In some methods of the invention, either a calpain inhibitor or an IGF1R inhibitor is contacted with a target cell or administered to a subject harboring the target cell. These methods are suitable for target cells for which that are currently no effective means for down-regulating LIN28 expression and promoting differentiation, e.g., cancer stem cells. Nevertheless, some preferred embodiments of the invention employ a dual inhibitor strategy for LIN28 suppression (as illustrated in FIG. 8). In these methods, small molecule inhibitors targeting both IGF1R and calpains are contacted with a target cell (e.g., a tumor cell or cancer stem cell) or administered to a subject afflicted with a tumor. The target cells can be treated with a combination of calpain inhibitor calpeptin (Cpep) and IGF1R inhibitor PPP, as exemplified herein. As exemplified herein, calpain inhibition rescues expression of dephosphorylated RBM3 in pluripotent cells. Also, as demonstrated in the Examples below, the combination of the two inhibitors led to advantageous and surprising therapeutic effects in reducing LIN28 levels in target cells (see, e.g., FIG. 4 and FIG. 7). The combination of these two classes of inhibitors could enhance the efficacy of chemotherapeutics administered concurrently (e.g., to make cancer stem cells more vulnerable to chemotherapy). Given the broad relevance of LIN28 in cancer and cancer stem cell viability, the combined use of IGF1R and calpain inhibitors to reduce LIN28 and promote let-7 biogenesis can also be efficacious as a neoadjuvant or adjuvant therapy in many cancers. Moreover, this combo therapy could induce death of tumor cells. The surprising effects obtained with the combo therapy of the invention are in contrast with the unsatisfactory results emerging from some clinical trials of IGF1R inhibitors for various cancers. Not intended to be bound in theory, the improved therapeutic activity of the dual inhibition could be due to that heightened calpain activity in some tumors negates the effect of IGF1R inhibitors on self renewal, pluripotency and tumorigenesis.

The novel methods of the invention can have broad therapeutic applications. For example, methods of the invention find therapeutic uses of stem cells through controlled differentiation. For example, with the present invention, inhibitors of IGF1R and calpain can be used as part of a strategy to guide the differentiation of hESCs and iPSCs in the context of regenerative treatment or medicinal uses of these cells (e.g., in subjects undergoing organ transplantations). Small molecule therapies inducing escape from pluripotency (tipping the “bi-stable switch”) could facilitate temporal and lineage control over introduced hESCs and iPSCs, or even endogenous stem cells, during disease or after injury.

Therapeutic applications of the invention also include its use in cancer therapy, where low level of dephosphorylated RBM3 and high LIN28 expression contribute critically to oncogenesis and to the viability of a subpopulation of “cancer stem cells” that are resistant to chemotherapy and underlie recurrence in many cancer types. The dual inhibitor therapy can also be used in conjunction with known antitumor drugs or treatment methods (e.g., other chemotherapeutic or immunotherapies for cancers). For example, it can be used together with surgery, chemotherapies or radiation therapies that have been routinely practiced in the art for the treatment of tumors. Thus, in some embodiments, subjects who have been undergoing surgical procedures to remove a tumor can be administered the dual inhibitors regimen of the invention to kill residual tumor cells and to prevent recurrence or metastasis. In some other embodiments, subjects in need of treatment for a tumor can be subject to the combination of a radiation therapy and the dual inhibitor therapy disclosed herein.

In some embodiments, subjects suffering from cancers can be simultaneously treated with the dual inhibitor regimen together with one or more chemotherapeutic agents, immunetherapeutic agents or other drugs. Such methods are particularly useful in inducing necrosis or apoptosis of cancer cell and/or cancer stem cells in subjects afflicted with LIN28 positive cancers. The combination therapy of the invention provides important advantages over existing therapies because administration of the dual inhibitors can enhance the tumor killing activity (by necrosis or apoptosis) of the co-administered chemotherapeutics or immunotherapeutic agents. There are many antineoplastic drugs and cytotoxic agents which can be readily utilized in combination with the dual inhibitor regimen described herein for treating tumors. Antineoplastic drugs include classes of agents such as alkylating agents, antimetabolites, antimitotics and topoisomerase II inhibitors. Specific examples include actinomycin, anthracyclines (e.g., doxorubicin, daunorubicin, Valrubicine, Idarubicine and epirubicin) and other cytotoxic antibiotics (e.g., bleomycin, plicamycin and mitomycin). In addition to these individual antitumor drugs, various chemotherapy regiment using two or more drugs can also be employed in combination with the dual inhibitor therapy disclosed herein. Detailed information about such chemotherapy regimens is readily available from, e.g., National Comprehensive Cancer Network (Jen Kintown, Pennsylvania). Various immunetherapeutic agents for treating cancer are also known and suitable for the combination therapy of the invention. These include, e.g., a number of therapeutic monoclonal antibodies have been approved for use in humans such as Avastin, Adcetris, Erbitux, Mylotarg, Zevalin, Vectibix, Rituxan, and Herceptin. Other immune therapies for treating cancer include cancer vaccines (e.g., Provenge® for prostate cancer), as well as non-specific immunetherapeutic agents such as cytokines, interleukins or interferons (e.g., IL-2, IL-7, IL-12, IL-21, IFNα and GM-CSF). Other agents that can be employed in the combination therapy of the invention include certain metabolic agents which are known to have therapeutic effects for cancer. For example, metformin has been shown to have a preventive effect in pancreatic cancer development.

The target cell suitable for the present invention can be any cell that expresses LIN28, RBM3, and/or another related target molecule. RBM3 is widely expressed in many tissues and cell types. These include cells from spleen, lung, intestine, brain, heart, liver, and other tissues. LIN28 is a pluripotency factor implicated in cell pluripotency, reprogramming, and oncogenesis. LIN28 is highly expressed embryonic stem cells and has been used to enhance the efficiency of the formation of induced pluripotent stem (iPS) cells from human fibroblasts. LIN28 is reactivated in ˜15% of all cancers, including those most frequent in the U.S. such as breast, prostate, and lung cancers. Moreover, high LIN28 expression defines a subset of so-called “cancer stem cells” that are thought to seed tumor formation and mediate recurrence in potentially all cancers. Methods of the present invention can be practiced with cells of any of these target cells. In some preferred embodiments, target cells in which reduced LIN28 level and/or up-regulated level of de-phosphorylated RBM3 is desired are tumor or cancer cells, human embryonic stem cells (hESCs), and induced pluripotent stem cells (iPSCs). In preferred embodiments, the methods of the invention are directed to cancer types that express high levels of LIN28, and to cancer stem cells (which may be present in all cancers) where LIN28 contributes to stemness and chemotherapeutic resistance.

Subjects afflicted with any cancer or tumor types may be treated with the therapeutic regimen described herein. The cancers and tumors suitable for treatment with compositions and methods of the present invention can be those present in a variety of tissues and organs. They also include cancer cells, tumor cells, which include malignant tumor cells, and the like that are found in the component cells of these tissues and/or organs. Examples include brain tumors (glioblastoma multiforme and the like), spinal tumors, maxillary sinus cancer, cancer of the pancreatic gland, gum cancer, tongue cancer, lip cancer, nasopharyngeal cancer, mesopharyngeal cancer, hypopharyngeal cancer, laryngeal cancer, thyroid cancer, parathyroid cancer, lung cancer, pleural tumors, cancerous peritonitis, cancerous pleuritis, esophageal cancer, stomach cancer, colon cancer, bile duct cancer, gallbladder cancer, pancreatic cancer, hepatic cancer, kidney cancer, bladder cancer, prostate cancer, penile cancer, testicular tumors, cancer of the adrenal glands, uterocervical cancer, endometrial cancer, vaginal cancer, vulvar cancer, ovarian cancer, ciliated epithelial cancer, malignant bone tumors, soft-tissue sarcomas, breast cancer, skin cancer, malignant melanomas, basal cell tumors, leukemia, myelofibrosis with myeloid metaplasia, malignant lymphoma tumors, Hodgkin's disease, plasmacytomas, and gliomas.

### IV. Inhibitors of IGF1R and Calpains

Various inhibitor or antagonist compounds that are specific for IGF1R or calpains can be employed in the practice of the present invention. Calpain inhibitors and IGF1R inhibitors of any chemical nature can be employed. These include, e.g., small organic compounds, antibodies or antigen-binding fragments (e.g., Fab, F(ab′)₂, Fd fragment, Fv fragment, dAb fragment or scFv), peptides or polypeptide agents, oligonucleotides or polynucleotide agents (including spiegelmers and aptamers), or other compounds. In some preferred embodiments, the invention employs calpain inhibitors and IGF1R inhibitors that are small organic compounds or antibodies (or antigenic fragments). Other than containing a moiety that inhibits IGF1R or calpains, the inhibitor compounds may additionally carry or be conjugated to additional moieties or agents with other activities, e.g., therapeutic or diagnostic functions. For example, the inhibitor compounds can harbor or be linked to active antitumor drug payloads or imaging agents.

In the case of IGF inhibitors, there are many compounds that are known to inhibit IGF1R. For example, specific IGF1R inhibitors have been developed by many pharmaceutical and biotech companies and are in various stages of clinical testing. Any of these compounds may be employed and/or modified in the practice of the present invention. Nevertheless, due to the similarity of the structures of IGF1R and the insulin receptor (IR), especially in the regions of the ATP binding site and tyrosine kinase regions, IGF1R inhibitors to be employed in the invention are preferably selective for IGF1R over IR. There are three main classes of inhibitors that have been shown to possess selectivity for IGF1R over IR. These include Tyrphostins such as AG538 (Blum et al., Biochemistry 39: 15705-12, 2000) and AG1024 (Ligeza et al., Acta. Biochim. Pol. 58: 391-396, 2011); pyrrolo(2,3-d)-pyrimidine derivatives such as AEW541 (Garcia-Echeverria et al., Cancer Cell 5:231-39, 2004); and monoclonal antibodies specific for IGF1R such as figitumumab (Haluska et al., Cancer Chemother. Pharmacol. 65:765-773, 2009). Additional IGF1R-specific inhibitors known in the art include picropodophyllin (PPP), Linsitinib (OSI-906), GSK1904529A, ADW742, GSK1838705A, BMS-536924, and BMS-754807. Chemical structures and biological properties (e.g., IGF1R-inhibiting function) of these compounds are also well documented in the art. See, e.g., Economou et al., Invest. Ophthalmol. Vis. Sci. 49:2337-42, 2008; Fox et al., Cancer Res. 71:6773-84, 2011; Kang et al., Cell Death Dis. 3(6): e336, 2012; Warshamana-Greene et al., Clin. Cancer Res. 11:1563-71, 2005; Sabbatini et al., Mol. Cancer Ther. 8(10):2811-20, 2009; Wang et al., Cancer Res. 172:59-76, 2007; Carboni et al., Mol Cancer Ther. 8:3341-9, 2009; and Shan et al., Hepatology 56:1004-14, 2012.

Similarly, uses of calpain inhibitors for targeting calpain enzymes in some indications are well known in the art. Many calpain inhibitors derived from both natural sources and chemical synthesis have been reported in the literature. See, e.g., Carragher et al., Curr. Pharm. Des. 12, 615-638, 2006; and Donkor et al., Curr. Med. Chem. 7, 1171-1188, 2000. These include bother peptide analogues and non-peptide. Peptidomimetic inhibitors are generally directed against the active site of calpain and can be subclassified into peptidyl epoxides, peptidyl aldehydes and peptidyl ketoamide classes (Carragher et al., Curr. Pharm. Des. 12, 615-638, 2006). Abbott Pharmaceuticals have disclosed novel carboxamide compounds with nanomolar potency against calpain 1 and high selectivity over cathepsins (see, e.g., WO 2010094755). Also, a unique class of non-peptide α-mercaptoacrylates, which do not target the active site of calpain but rather interact with the regulatory calcium-binding domain, demonstrated high selectivity for calpains and highlight the potential for developing allosteric calpain inhibitors. These compounds are described in the art, e.g., Todd et al., J. Mol. Biol. 328, 131-146, 2003; and Wang et al. Proc. Natl. Acad. Sci. USA 93, 6687-6692, 1996.

In some embodiments of the invention, several specific calpain inhibitors known in the art can be employed. These include calpeptin (Cpep), ALLN (aka Calpain Inhibitor I), MDL2817 (Calpain Inhibitor III), PD150606, SJA6017, AK275, ABT-705253, and SNJ-1945. The chemical structures, inhibitory activities and other properties of these compounds are described in the art. See, e.g., Ebisui et al., Biochem. Mol. Biol. Int. 32:515-21, 1994; Sarah et al., Nature Reviews Cancer 11, 364-374, 2011; Graybill et al., Bioorg. Med. Chem. Lett. 5, 387-392, 1995; Mehdi et al., Biochem. Biophys. Res. Commun. 157:1117-23, 1988; Wang et al., Proc. Natl. Acad. Sci. U.S.A. 93: 6687-92, 1996; Kupina et al., J. Neurotrauma 18: 1229-40, 2001; Lubisch et al., J. Med. Chem. 46: 2404-12, 2003; Nimmrich et al., Br. J. Pharmacol. 159: 1523-31, 2010; and Koumura et al., Neuroscience 157: 309-18, 2008. Some of these inhibitors target the catalytic domain of calpain, e.g., SJA6017, ALLN, AK275 ketoamide and carboxamide. Some other compounds are allosteric inhibitors of calpain, e.g., PD150606, quinazolinecarboxamides, and quinazolinecarboxamides.

The various inhibitor compounds exemplified herein and their variants or derivatives can all be readily obtained from commercial suppliers (e.g., Sigma-Aldrich, St. Louis, Mo.) or de novo synthesized using routinely practiced methods of organic chemistry. For example, relative to one of the specific inhibitor compounds exemplified herein, some of their derivative compounds can have one or more mono- or multi-valent groups replaced with a different mono- or multi-valent group. The replaced group can be, e.g., H; halogen; straight, cyclic or branched chain alkyl; straight, cyclic or branched chain alkenyl; straight, cyclic or branched chain alkynyl; halo-alkyl, -alkenyl or -alkynyl; CN; CF₃; aryl and substituted aryl groups in which any or all H groups of the aryl ring is substituted with a different group; heterocyclic and substituted heterocyclic groups in which any or all groups of the aryl ring is substituted with a different group; carboxyl; carbonyl, alkoxyl; alkyloxyalkanes; alkoxycarbonyl; aryloxyl, heterocyclyloxyl; hydroxyl; amine; amide; amino; quaternary amino; nitro; sulfonyl; alkylamine; silyl, siloxyl; saturated C—C bonds; unsaturated C—C bonds; ester, ether, amino; amide, urethane, carbonyl, acetyl and ketyl groups; hetero atoms, including N, S and O; polymer groups; and amino acids. In some derivative compounds, one or more hydrogens can be substituted with a lower alkyl group. The various derivative compounds can be subject to a functional test (e.g., proliferation inhibition assay as exemplified herein) to ascertain their inhibitory activities.

In addition to the specific calpain inhibitor and IGF1R inhibitor compounds exemplified herein, novel calpain inhibitors or IGF1R inhibitors that can be identified and produced via routinely practiced methods may also be used in the methods of the invention. For example, derivative compounds that can be easily synthesized from these compounds may also be suitable for the practice of the methods of the invention. Typically, variants or derivative compounds with similar or improved properties can be obtained by rational optimization of the exemplified inhibitor compounds (the lead compounds). Optionally, the compounds generated via rational design can be further subjected to a functional test or screening in order to identify compounds with improved activities. Methods for designing and screening such variant compounds are well known to the skilled artisans in the art. For example, calpastatin is the ubiquitously expressed endogenous inhibitor of μ-calpain and m-calpain. It consists of an N-terminal L domain that contains an N-terminal XL region, and four repetitive inhibitory domains (I-IV). The structural basis and mechanism of calpain inhibition by its highly specific endogenous inhibitor calpastatin provides a template for the development of novel calpain intervention strategies that target the active site cleft and/or the non-catalytic domains. In some embodiments, combinatorial libraries of small molecule candidate agents can be employed to screen for novel small molecule inhibitors of IGF1R or calpains. A number of specific assays are available for such screening, e.g., as described in Schultz et al., Bioorg. Med. Chem. Lett. 8:2409-2414, 1988; Weller et al., Mol Divers. 3:61-70, 1997; Fernandes et al., Curr. Opin. Chem. Biol. 2:597-603, 1998; and Sittampalam et al., Curr. Opin. Chem. Biol. 1:384-91, 1997. Various biochemical and molecular biology techniques or assays well known in the art can be employed to practice the screening methods of the present invention. Such techniques are described in, e.g., Handbook of Drug Screening, Seethala et al. (eds.), Marcel Dekker (1^{st} ed., 2001); High Throughput Screening: Methods and Protocols (Methods in Molecular Biology, 190), Janzen (ed.), Humana Press (1^{st} ed., 2002); Current Protocols in Immunology, Coligan et al. (Ed.), John Wiley & Sons Inc (2002); Sambrook et al., Molecular Cloning: A Laboratory Manual, Cold Spring Harbor Press (3^{rd} ed., 2001); and Brent et al., Current Protocols in Molecular Biology, John Wiley & Sons, Inc. (ringbou ed., 2003).

### V. Pharmaceutical Compositions and Administration

To practice methods of the invention, a calpain inhibitor and/or an IGF1R inhibitor, or a pharmaceutical composition containing the compound(s) can be contacted with the target cell via any appropriate means. The target cell can be present in vitro, e.g., a cell sample obtained from a subject. It can also be present in vivo in a subject. Thus, in some methods, the inhibitor compounds are contacted with a target cell in vitro using a cultured cell line or ex vivo with cells isolated from a subject. In some other methods, the compounds or pharmaceutical composition are contacted with the target cell in vivo.

The calpain inhibitor and IGF1R inhibitor and the other therapeutic agents disclosed herein can be directly contacted with a target cell or administered to a subject in need of treatment. However, these therapeutic compounds are preferably administered in pharmaceutical compositions which comprise the inhibitors and/or other active agents along with a pharmaceutically acceptable carrier, diluent or excipient in unit dosage form. Accordingly, the invention provides pharmaceutical compositions comprising one or more of the inhibitor compounds disclosed herein. The invention also provides a use of the described calpain inhibitors and IGF1R inhibitors in the preparation of pharmaceutical compositions or medicaments for treating the above described diseases or medical disorders. Therapeutic kits which comprise at least one inhibitor compound described herein and an instruction sheet for using the compound to treat atopic allergies are also provided in the invention.

Pharmaceutically acceptable carriers are agents which are not biologically or otherwise undesirable. These agents can be administered to a subject along with an inhibitor compound without causing any undesirable biological effects or interacting in a deleterious manner with any of the components of the pharmaceutical composition. The compositions can additionally contain other therapeutic agents that are suitable for treating inflammatory disorders. Pharmaceutically carriers enhance or stabilize the composition or facilitate preparation of the composition. Pharmaceutically acceptable carriers include solvents, dispersion media, coatings, antibacterial and antifungal agents, isotonic and absorption delaying agents, and the like that are physiologically compatible. The pharmaceutically acceptable carrier employed should be suitable for various routes of administration described herein. Additional guidance for selecting appropriate pharmaceutically acceptable carriers is provided in the art, e.g., Remington: The Science and Practice of Pharmacy, Mack Publishing Co., 20^{th} ed., 2000.

Pharmaceutical compositions of the invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for molecules of the invention include ethylene-vinyl acetate copolymer particles, osmotic pumps, implantable infusion systems, and liposomes. Formulations for inhalation may contain excipients, for example, lactose, or may be aqueous solutions containing, e.g., polyoxyethylene-9-lauryl ether, glycocholate and deoxycholate, or may be oily solutions for administration in the form of nasal drops, or as a gel.

The calpain inhibitor and IGF1R inhibitor for use in the methods of the invention should be administered to a subject in an amount that is sufficient to achieve the desired therapeutic effect (e.g., eliminating or ameliorating symptoms associated with a cancer) in a subject in need thereof. Typically, a therapeutically effective dose or efficacious dose of the inhibitor is employed in the pharmaceutical compositions of the invention. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular compositions of the present invention employed, the route of administration, the time of administration, and the rate of excretion of the particular compound being employed. It also depends on the duration of the treatment, other drugs, compounds and/or materials used in combination with the particular compositions employed, the age, gender, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. Typically, a pharmaceutically effective dosage would be between about 0.001 and 100 mg/kg body weight of the subject to be treated.

The inhibitor compounds and other therapeutic regimens described herein are usually administered to the subjects on multiple occasions. Intervals between single dosages can be daily, weekly, or even monthly. Dosage and frequency vary depending on the half-life of the inhibitor compound and the other drugs in the subject. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the subject can be administered a prophylactic regimen.

A pharmaceutical composition containing an inhibitor compound described herein and/or other therapeutic agents can be administered by a variety of methods known in the art. The routes and/or modes of administration vary depending upon the desired results. Depending on the route of administration, the active therapeutic agent may be coated in a material to protect the compound from the action of acids and other natural conditions that may inactivate the agent. Conventional pharmaceutical practice may be employed to provide suitable formulations to administer such compositions to subjects. Any appropriate route of administration may be employed, for example, but not limited to, intravenous, parenteral, transcutaneous, subcutaneous, intramuscular, intracranial, intraorbital, intraventricular, intracapsular, intraspinal or oral administration. Depending on the specific conditions of the subject to be treated, either systemic or localized delivery of the therapeutic agents may be used in the treatment. In some embodiments, the therapeutic composition is administered to the subject via systemic route, e.g., by injection. In some other embodiments, the composition is administered to the subject via local administration, e.g., topical application or inhalation.

In some embodiments, the present invention provides a packaged pharmaceutical composition for treating or preventing atherosclerosis such as a kit or other container. Typically, the kit or container holds a therapeutically effective amount of each of a calpain inhibitor and an IGF1R inhibitor. The kit can optionally contain an instruction sheet detailing how to use the dual inhibitors in combination to down-regulate in a target cell (e.g., tumor cell or stem cell) the level of LIN28 and/or the level of phosphorylated RBM3.

### EXAMPLES

The following examples are provided to further illustrate the invention but not to limit its scope. Other variants of the invention will be readily apparent to one of ordinary skill in the art and are encompassed by the appended claims.

### Example 1. Regulation of Let-7 Family miRNA Biogenesis by RBM3

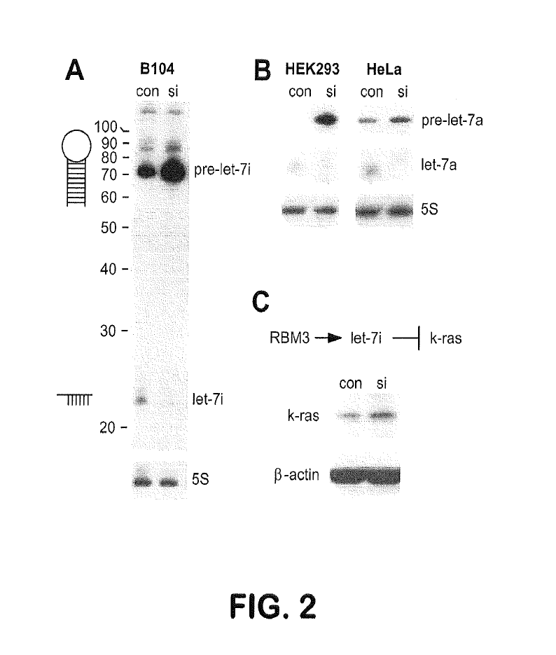

RNA-binding motif protein 3 (RBM3) promotes miRNA biogenesis at the Dicer step by binding to pre-miRNAs and derepressing their access to Dicer. It has been shown that RBM3 strongly promotes the biogenesis of all members of the let-7 family, as well as 3 other miRNAs that, like let-7, downregulate LIN28 (i.e., miRs 9, 30, 125). In all cancer cell lines tested so far, let-7 biogenesis is abolished in the absence of RBM3 and promoted by RBM3 overexpression—i.e. let-7 production shows a strong dependence on RBM3. As shown in FIG. 2, we observed a profound effect of RBM3 knockdown on let-7 processing and let-7 target expression. Taken with the fact that RBM3 is highly expressed in proliferating and differentiating cell fields of mouse brain, this finding prompted us to study RBM3's role in differentiation. Importantly, we found that manipulation of RBM3 levels in the P19 embryonal carcinoma cell line and in mouse ESCs regulates LIN28 expression in a manner consistent with RBM3's effects on let-7 biogenesis (see below). These and other data indicate that RBM3 can push the LIN28-let7 “bi-stable switch” toward let-7 biogenesis and differentiation. The possibility of a therapeutically viable method for manipulating LIN28 levels via RBM3 was rendered by subsequent studies we conducted on the phosphorylation of RBM3 (see below).

### Example 2. Phosphoregulation of RBM3

Phosphorylation of RBM3 on tyrosines proximal to the C-terminus has been identified in hundreds of phosphoproteomic screens of solid tumors, hematopoietic malignancies, and cancer cell lines (www.phosphosite.org). Our mutational analyses of 4 C-terminal tyrosines confirmed that these residues are phosphorylated. We have found that a dephosphorylated form of RBM3 preferentially associates with pre-miRNAs (data not shown), suggesting that it is this form that promotes miRNA biogenesis. Interestingly, a single non-phosphorylatable mutant, Y146F, induced dephosphorylation of endogenous RBM3 in the B104 cell line, presumably by competing for its endogenous kinase. Bioinformatic analysis predicted that Y146 is a strong consensus site for the Insulin Like Growth Factor I Receptor (IGF1R); indeed, the sequence surrounding Y146 is perfectly homologous to a site in PDK1 known to be phosphorylated by IGF1R. IGF1R is a receptor tyrosine kinase that is involved in the maintenance of pluripotency and has been implicated in the risk, pathogenesis, and prognosis of many cancers. Our study suggests that phosphorylation of RBM3 by IGF1R contributes to pluripotency and oncogenesis by blocking RBM3-mediated enhancement miRNAs that target LIN28. It is notable in this regard that IGF1R expression is downregulated by let-7, suggesting that a double negative feedback loop exists analogous to the LIN28-let-7 interaction.

### Example 3. A Dephosphorylated Form of RBM3 is not Expressed in Pluripotent Cells

Consistent with the above study, we found that non-phosphorylatable mutants of RBM3 (compared to wild-type) are difficult to express in pluripotent P19 cells, and that both P19 cells and other cancer cell lines contain very low levels of the dephosphorylated form of endogenous RBM3. Our data raise the possibility that IGF1R inhibitors may promote let-7 formation by blocking the Tyr phosphorylation of RBM3, which would have anti-cancer effects. Thus far, clinical data on IGF1R inhibitors have been mixed (Yee et al., J. Natl. Cancer Inst. 104: 975-981, 2012). As we describe below, we believe this is due in part to a second regulatory mechanism that prevents dephosphorylated RBM3 from accumulating in pluripotent and cancerous cells.

### Example 4. Calpain Inhibition Rescues Expression of Dephosphorylated RBM3

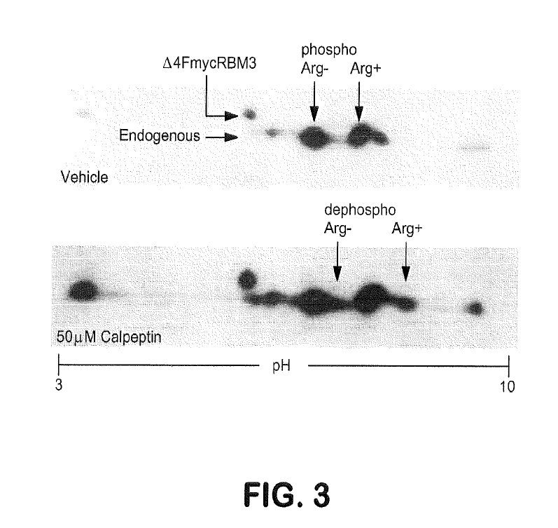

We further observed that calpain inhibition rescues expression of dephosphorylated RBM3 in pluripotent cells. Calpains are Ca⁺⁺-activated cysteine proteases that regulate diverse cellular processes. Calpain's role in pluripotent and cancerous cells prompted us to test whether it regulates the expression of the dephosphorylated form of RBM3 in pluripotent cells (and accounts for the difficulty in expressing non-phosphorylatable mutants in these cells). We found that addition of the calpain inhibitor calpeptin increased levels of a non-phosphorylatable mutant of RBM3, as well as the endogenous dephosphorylated form of RBM3 that binds pre-miRNAs (dephos Arg+; FIG. 3). These data suggest that inhibition of calpain can reduce LIN28 levels by allowing the expression of a form of RBM3 that promotes biogenesis of let-7 and other miRNAs.

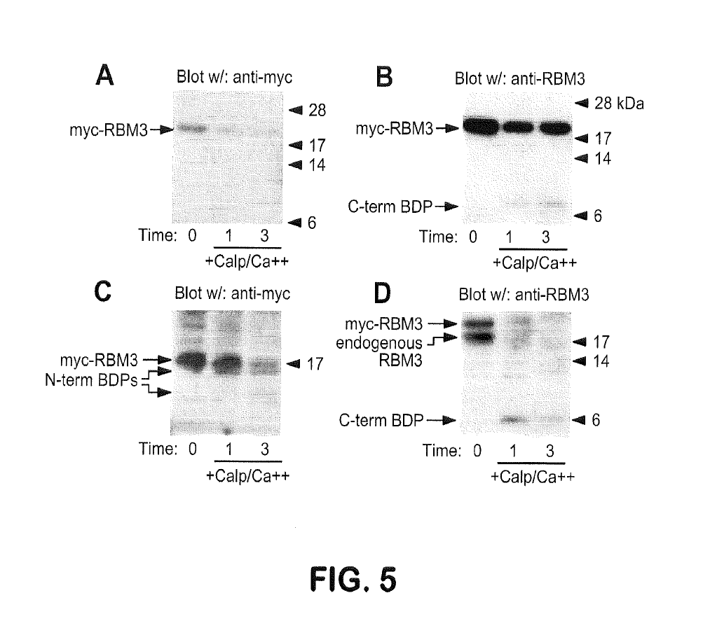

The above data and observations from studies in P19 cells are supported by in vitro studies of calpain-mediated cleavage of RBM3. We tested the ability of purified calpain 1 to cleave myc-RBM3 in two experimental systems: one using purified recombinant myc-RBM3 and the other using lysates of B104 cells that expressed myc-RBM3 (FIG. 5). The results in each case show preferential cleavage of the c-terminus. Within the resolution limits of the gels used, an ˜6 kDa c-terminal breakdown product (BDP) is evident using our anti-RBM3 antibody that was raised to a peptide corresponding to the last 18 c-terminal amino acids of RBM3. Using anti-myc tag antibodies, which recognize the N-terminal myc epitope tag, it is evident that there are also smaller C-terminal cleavages because N-terminal BDPs appear that are slightly under ˜17 kDa.

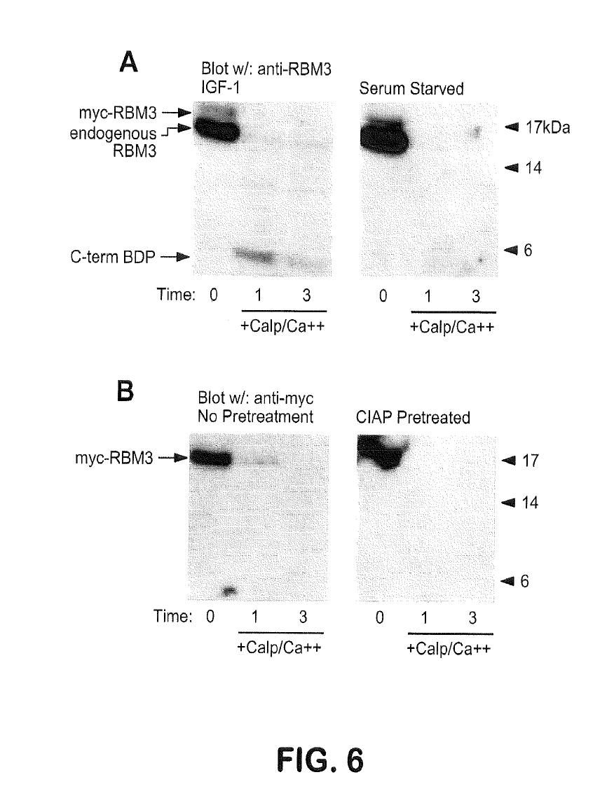

In a separate experiment, we assessed the effect of stimulating IGF1R on RBM3's susceptibility to cleavage by calpain (FIG. 6). While the baseline rate of cleavage was faster in this experiment compared to FIG. 5 (as seen in the control condition), addition of IGF1 to B104 cells prior to lysis and incubation with purified calpain reduced this cleavage. In a second approach, we treated purified myc-RBM3 with a phosphatase (CIAP) prior to its incubation with calpain. This resulted in a more rapid cleavage by calpain 1 compared to no pretreatment (FIG. 6B). Optimized in vitro conditions are determined for calpain-mediated cleavage (to obtain a slower rate) and both IGF1 and phosphatase treatments to better titrate the effects of phosphorylation on RBM3 cleavage. These data support the model we arrived at from in situ studies using P19s: i.e., that phosphorylation of RBM3 by IGF1R regulates its cleavage by calpain.

Taken together, it appears that levels of dephospho-RBM3 are kept low in stem cells and cancers by two signaling enzymes that have been implicated in the maintenance of pluripotency and oncogenesis: IGF1R and calpains. We believe this is to minimize biogenesis of let-7 and other miRNAs that target LIN28 and regulate cell differentiation.

### Example 5. Inhibitors of IGF1R and Calpain Reduce LIN28 Levels

As predicted from RBM3's role in miRNA biogenesis, we found that knockdown of RBM3 leads to large increases in LIN28 levels in P19 and mouse ESCs (not shown), and even blocks the reduction in LIN28 induced by retinoic acid (RA) applied at the neurosphere stage to differentiate P19 cells into neurons (FIG. 4A). In this neurosphere stage, RA normally induces upregulation and, importantly, dephosphorylation of RBM3 (not shown). Thus, knockdown of RBM3 is expected to reduce the dephosphorylated form of RBM3 that promotes let-7 biogenesis and downregulation of LIN28. As shown above, RBM3's ability to promote the biogenesis of let-7 and other miRNAs that reduce LIN28 expression is also regulated by calpain, which degrades the dephosphorylated form of RBM3. Dual inhibition of these two enzymes, IGF1R and calpain, which are involved in the maintenance of pluripotency and cancer, can promote let-7 biogenesis and reduce LIN28 levels, likely synergistically in combination. Indeed, we found that acute (˜12 hrs) inhibition of IGF1R and calpain in P19 cells leads to reduced LIN28 expression (FIG. 7B; FIG. 4B).

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

1. A method for inhibiting phosphorylation, downregulating cellular level of phosphorylated form, or upregulating cellular level of dephosphorylated form, of mRNA-binding protein 3 (RBM3) in a target cell, comprising contacting the target cell with (1) a calpain inhibitor or (2) an IGF1R inhibitor, thereby inhibiting phosphorylation, down-regulating phosphorylated RBM3 or upregulating de-phosphorylated RBM3 in the target cell.

2. The method of claim 1, wherein the target cell is a cancer cell, a cancer stem cell, or a stem cell.

3. The method of claim 2, wherein the stem cell is hESC or iPSC.

4. The method of claim 1, wherein the target cell is present in a subject.

5. The method of claim 1, wherein the target cell is contacted with both a calpain inhibitor and an IGF1R inhibitor.

6. The method of claim 1, wherein the calpain inhibitor and the IGF1R inhibitor are small molecule compounds, antibodies or antigenic fragments.

7. The method of claim 1, wherein a target cell is further contacted with chemotherapeutic agent, an immunotherapeutic agent, or a metabolic agent.

8. A method for down-regulating expression or cellular level of pluripotency factor LIN28 in a target cell, comprising contacting the target cell with (1) a calpain inhibitor or (2) an IGF1R inhibitor, thereby down-regulating expression or cellular level of pluripotency factor LIN28 in the target cell.

9. The method of claim 8, wherein the target cell is a cancer cell, a cancer stem cell, or a stem cell.

10. The method of claim 9, wherein the stem cell is hESC or iPSC.

11. The method of claim 8, wherein the target cell is present in a subject.

12. The method of claim 8, wherein the target cell is contacted with both a calpain inhibitor and an IGF1R inhibitor.

13. The method of claim 8, wherein the target cell has shown reactivation of the Lin28 gene.

14. The method of claim 8, wherein the calpain inhibitor and the IGF1R inhibitor are small molecule compounds, antibodies or antigenic fragments.

15. The method of claim 8, wherein the target cell is further contacted with a chemotherapeutic agent, an immunotherapeutic agent, or a metabolic agent.

16. A method for treating or inhibiting the growth of a cancer in a subject, comprising administering to a subject in need of treatment a pharmaceutical composition comprising (1) a calpain inhibitor and/or (2) an IGF1R inhibitor, thereby treating or inhibiting the growth of a cancer in the subject.

17. The method of claim 16, wherein the administered calpain inhibitor and IGF1R inhibitor are small molecule compounds, antibodies or antigenic fragments.

18. The method of claim 16, wherein the subject is administered both a calpain inhibitor and an IGF1R inhibitor.

19. The method of claim 16, wherein the calpain inhibitor and IGF1R inhibitor are administered in conjunction with one or more chemotherapeutic agents, immunotherapeutic agents, or metabolic agents.

20. The method of claim 19, wherein the calpain inhibitor and the IGF1R inhibitor are administered prior to, simultaneously with, or subsequent to the chemotherapeutic agents, immunotherapeutic agents, or metabolic agents.

21-30. (canceled)
